(19) United States
(12) Patent Application Publication
(10) Pub. No.: US 2016/0300472 A1
(43) Pub. Date: Oct. 13, 2016

(54) Patient Monitoring System and Apparatus

(71) Applicant: AvaSure Holdings, Inc., Belmont, MI (US)

(72) Inventors: Walter Eadelman, Belmont, MI (US); Brian Meinke, Rockford, MI (US); Stacey Overholt, Ada, MI (US); Doreen DalSanto, Grand Rapids, MI (US)

(21) Appl. No.: 15/188,789

(22) Filed: Jun. 21, 2016

Related U.S. Application Data
(63) Continuation of application No. 14/254,694, filed on Apr. 16, 2014.

(51) Int. Cl. G08B 21/22; G08B 21/02; G08B 5/22; H04N 5/445; H04W 4/00; H04N 7/18; G06F 3/16; G10L 15/26; A61B 5/11; H04B 5/00

(52) U.S. Cl. CPC G08B 21/22; A61B 5/1113; G08B 21/02; G08B 5/22; H04B 5/0062; H04W 4/008; H04N 7/181; G06F 3/16; G10L 15/265; H04N 5/44504; A61B 2505/09; A61B 2505/05

(57) ABSTRACT

A system and apparatus for monitoring a patient, the apparatus including an image capturing device; an electronic input device; a display device; and a controller in electronic communication all three devices; wherein the controller is configured to receive an electronic signal encoded with an image of a patient room from the image capturing device, display the image of the patient room on the display device, receive, via the electronic input device, an electronic input signal regarding a status associated with a patient in the patient room, and display a message on the display device based on the status of the patient.

### TECHNICAL FIELD

This disclosure relates generally to patient monitoring.

### BACKGROUND

A patient room may be equipped with a video camera. A video feed of the patient room is displayed on a monitor in an operator room, where an operator can monitor the patient by viewing the video feed displayed on the monitor. The monitor can display multiple video feeds from different patient rooms, thereby allowing the operator to monitor several patients from the operator room. If a patient does something that is inconsistent with the patient's medical condition, then the operator can alert a healthcare provider. For example, if a patient leaves the patient room without authorization, then the operator can alert a healthcare provider. Sometimes the operator may inadvertently provide false alerts by alerting that a patient is missing but in actuality the patient may have been properly removed from the patient room by a healthcare provider. Therefore, there is a need to reduce false alerts in patient monitoring.

### SUMMARY

An aspect of the disclosure provides an apparatus for monitoring a patient. The apparatus includes an image capturing device, an electronic input device, a display device and a controller. The controller is in electronic communication with the image capturing device, the electronic input device and the display device. The controller is configured to receive an electronic signal encoded with an image of a patient room from the image capturing device, display the image of the patient room on the display device, receive, via the electronic input device, an electronic input signal regarding a status associated with a patient in the patient room, and display a message on the display device based on the status associated with the patient.

Implementations of the disclosure may include one or more of the following features. In some implementations, the electronic input signal regarding the status associated with the patient includes a time identifier and the message displayed on the display device includes the time identifier. In other implementations, the electronic input signal regarding the patient includes a location and the message displayed on the display indicates the location. In yet other implementations, the electronic input signal regarding the patient includes a status of the patient and the message displayed on the display indicates the status of the patient. The status of the patient may include a medical condition or a restriction of the patient.

In some implementations, the electronic input device authenticates the healthcare provider. The electronic input device may authenticate the healthcare provider through a RFID (Radio Frequency Identification) badge, a NFC (Near Field Communications) tag, a password, a fingerprint, an eye scan or a voice command.

In some implementations, the electronic input device includes a microphone and the electronic input signal includes audio captured by the microphone. In other implementations, the electronic input device may include a mobile electronic communication device such as a cell phone, a smart phone or a tablet computer. In yet other implementations, the electronic input device may include a computer terminal such as a personal computer (PC). The electronic input device may include a button that can be depressed by a healthcare provider to indicate that the healthcare provider is removing a patient from a patient room. The electronic input device may include a RFID reader that can read a RFID badge.

The apparatus may include an input datastore that stores a plurality of electronic input signals and wherein the electronic input device receives a selection of one of the plurality of electronic input signals as the electronic input signal regarding the patient. In some implementations, the apparatus compares the electronic input signal received from the electronic input device with the plurality of electronic input signals stored in the input datastore to identify the electronic input signal. The apparatus may also include a message datastore that stores a plurality of messages, each message corresponds with one or more inputs and the controller displays the message that corresponds with the input received from the healthcare provider.

Another aspect of the disclosure provides a method for monitoring a patient, the method includes receiving, at a controller, an electronic signal encoded with an image of a patient room from an image capturing device. The image of the patient room is displayed on a display device. The method further includes receiving, via an electronic input device, an electronic input signal regarding a patient in the patient room from a healthcare provider. The method also includes displaying a message on the display device based on the electronic input signal received from the healthcare provider.

In some implementations, the method includes receiving a time identifier as the electronic input signal regarding the patient and displaying the time identifier as a part of the message on the display device. In other implementations, the method includes receiving a location as the electronic input signal regarding the patient and displaying an indicium of the location as a part of the message on the display device. In yet other implementations, the method includes receiving a status of the patient as the electronic input signal regarding the patient and displaying an indicium of the status as a part of the message on the display device. The method may include receiving a medical condition or a restriction as the status of the patient. The method may further include receiving a termination time indicating when the medical condition or restriction will cease to exist.

In some implementations, the method includes authenticating the healthcare provider. The method may include reading, via a RFID reader, a RFID badge of a healthcare provider. The method may include receiving a password, a fingerprint, a voice command or an eye scan to authenticate the healthcare provider.

In some implementations, the electronic input device includes a microphone and the method includes receiving audio input, via the microphone, as the electronic input signal regarding the patient. In other implementations, the electronic input device includes a mobile electronic communication device and the method includes receiving the electronic input signal via the mobile electronic communication device. In yet other implementations, the electronic input device includes a RFID reader and the method includes reading a RFID badge via the RFID reader. In further implementations, the electronic input device includes a computer terminal and the method includes receiving the electronic input signal via the computer terminal.

In some implementations, the method further includes storing a plurality of electronic input signals in an input datastore and receiving a selection of one of the plurality of electronic input signals as the electronic input signal regarding the patient. The method may include comparing the received electronic input signal with the plurality of stored electronic input signals to identify the received input. The method may include displaying the plurality of electronic input signals on the electronic input device and receiving a selection of one of the plurality of electronic input signals.

In some implementations, the method includes storing a plurality of messages in a message datastore, each message corresponds with one or more electronic input signals. The method further includes determining whether one of the plurality of messages corresponds with the electronic input signal received from the healthcare provider and displaying the message that corresponds with the electronic input signal received from the healthcare provider.

Yet another aspect of the disclosure provides an apparatus for monitoring a patient, the apparatus includes a video camera, a microphone, a monitor, a message datastore storing a plurality of messages and a controller. The controller is in electronic communication with the video camera, the microphone and the monitor. The controller is configured to receive a video feed of a patient room from the video camera, display the video feed of the patient room on the monitor, receive, via the microphone, an electronic input signal regarding a patient in the patient room from a healthcare provider, select, from the message datastore, one of the plurality of messages corresponding with the electronic input signal, and display the selected message on the monitor in association with the video feed of the patient room being displayed on the monitor. In some implementations, the selected message is overlaid on top of the video feed of the patient room being displayed on the monitor. The display may display a plurality of video feeds, each video feed being from a different patient room.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system for monitoring a patient.

FIG. 2 is a block diagram of the example system shown in FIG. 1.

FIG. 3 is an example arrangement of operations for monitoring a patient.

FIG. 4 is a block diagram of another example system for monitoring a patient.

FIG. 5 is another example arrangement of operations for monitoring a patient.

FIG. 6 is a block diagram of yet another example system for monitoring a patient.

FIG. 7 is yet another example arrangement of operations for monitoring a patient.

FIG. 8 is a diagram of example messages displayed on a display.

Like reference symbols in the various drawings indicate like elements.

### DETAILED DESCRIPTION

In this document, a “healthcare provider” may include a physician, a nurse, a technician, a volunteer, a clergy or other staff. The “healthcare provider” may be a licensed individual, for example a registered nurse or a licensed physician. Alternatively, the “healthcare provider” may be a non-licensed individual, for example a family member providing care to a patient or a visitor visiting the patient. The “healthcare provider” may also include the patient itself or another patient sharing a room with the patient. This definition of the “healthcare provider” is non-limiting and may encompass other individuals or entities, for example a robot.

A “status” of the patient may include a diagnosis of the patient, protected health information of the patient, a description of the health of the patient, restrictions imposed on the patient or a location of the patient. The “status” may include a specific location of the patient, for example rehabilitation center, physical therapy, surgery room, etc. Alternatively, the “status” may indicate that the patient is not in the patient room without indicating the specific location of the patient. The “status” may also indicate whether the removal of the patient from the patient room was authorized or not authorized by the healthcare provider.

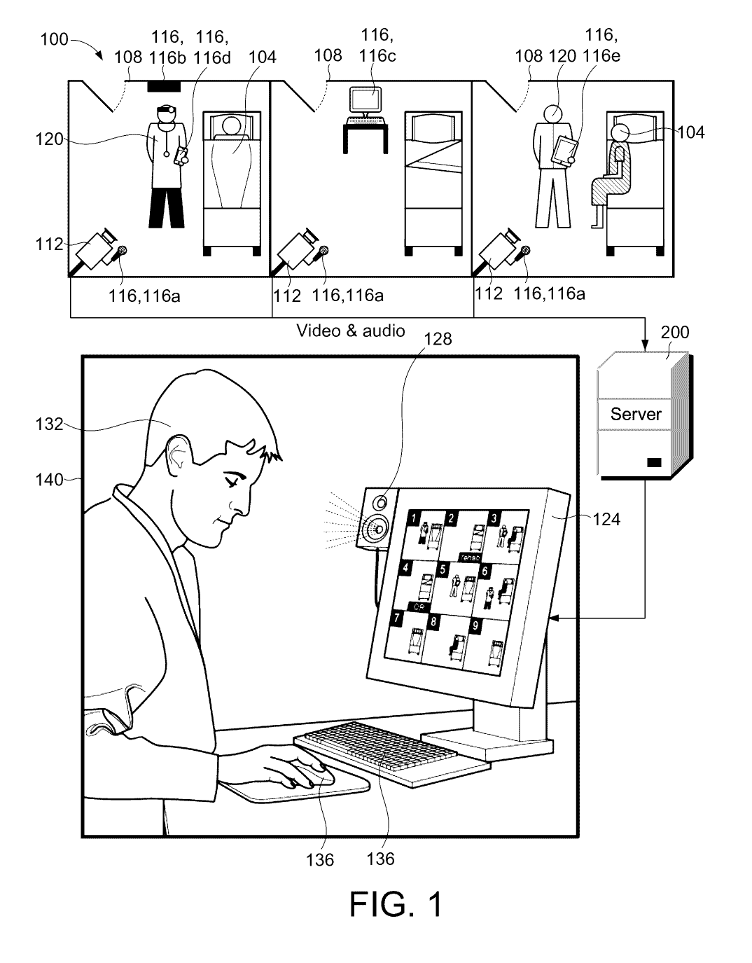

FIG. 1 illustrates a system 100 for monitoring a patient 104. The patient 104 resides in a patient room 108. The patient room 108 is equipped with an image capturing device 112 and an electronic input device 116. The image capturing device 112 may include a camera, a video camera, a charge coupled device (CCD), or the like. The electronic input device 116 may include a microphone 116A, a RFID scanner 116B, a computer terminal 116C, a mobile phone 116D, a tablet computer 116E, or a touchscreen display. The mobile phone 116D and the tablet computer 116E may belong to a healthcare provider 120. The image capturing device 112 captures images or videos of the patient room 108. The microphone 116A captures audio from the healthcare provider 120. The system 100 includes a display device 124 and a speaker 128. The display device 124 may include a liquid crystal display (LCD), a light emitting diode (LED) display, an organic light emitting diode (OLED) display, or the like.

The display device 124 and the speaker 128 are located in an operator room 140. The display device 124 displays images or videos captured by the image capturing device 112. The speaker 128 outputs audio captured by the microphone 116A. An operator 132 can view the display device 124 to get a view of the patient room 108. The operator 132 can listen to audio being outputted from the speaker 128 to listen to the healthcare provider 120 speaking through the microphone 116A in the patient room 108.

The system 100 includes a server 200. The server 200 receives an electronic input signal encoded with a video or an image from the image capturing device 112 located in the patient room 108. The server 200 sends the video or images received from the image capturing device 112 to the display device 124 in the operator room 140. The server 200 receives audio from the microphone 116A in the patient room 108 and sends the audio to the speaker 128 in the operator room 140.

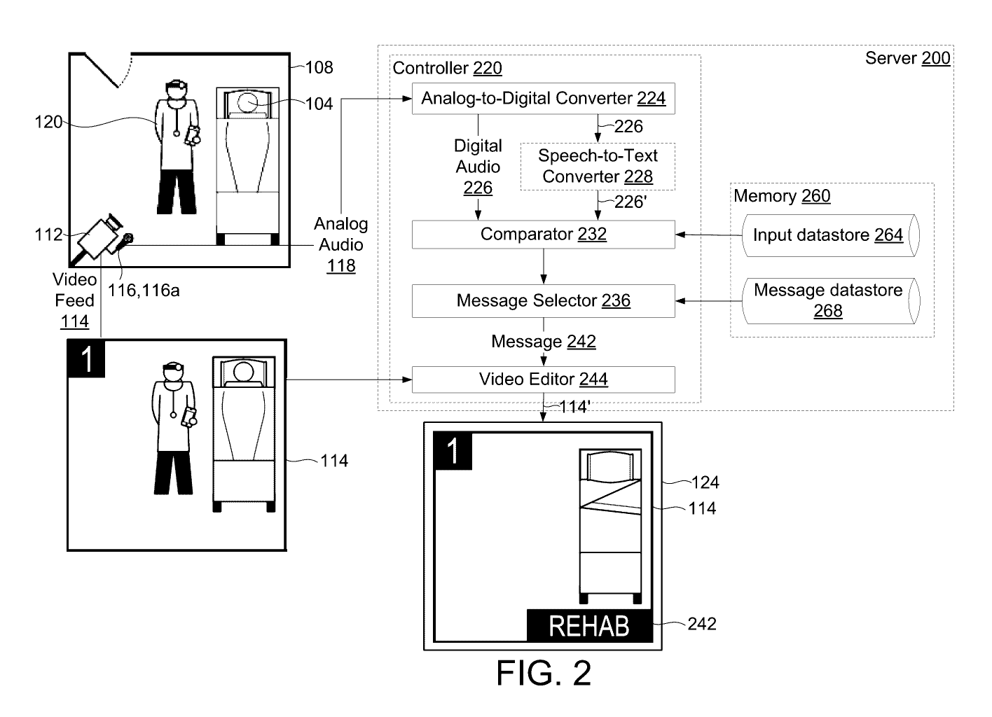

FIG. 2 shows an example implementation of the server 200. The server 200 includes a controller 220 and a memory 260. The controller 220 includes an analog-to-digital converter (ADC) 224, a speech-to-text converter 228, a comparator 232, a message selector and a video editor 244. The memory 260 includes an input datastore 264 and a message datastore 268.

The controller 220 receives analog audio 118 from the microphone 116A in the patient room 108. The ADC 224 may convert the analog audio 118 to digital audio 226 by sampling or periodically measuring the amplitude of the analog audio 118. The sampling rate of the ADC 224 may be varied to achieve a desired level of audio quality. The ADC 224 may employ a filter to remove any background noise from the patient room 108. The ADC 224 may add a low-amplitude white noise to the input to improve the performance of the ADC 224 when the amplitude of the analog audio 118 is very low. In the example implementation of FIG. 2, the ADC 224 is shown as part of the controller 220. However, in alternative implementations, the ADC 224 may be included in the image capturing device 112.

A speech-to-text converter 228 may convert the digital audio 226 into text 226′. The speech-to-text converter 228 may use speaker-independent speech recognition. Alternatively, the speech-to-text converter 228 may use speaker-dependent speech recognition in which the healthcare provider 120 reads sections of predetermined text into the microphone 116A to train the speech-to-text converter 228. The accuracy of the speech-to-text converter 228 may be improved by using speaker-dependent speech recognition. The accuracy of the speech-to-text converter 228 may also be improved by using a vocabulary and by limiting the size of the vocabulary. The speech-to-text converter 228 may use a speech recognition system that is based on Hidden Markov Models. Other models are also contemplated, for example Dynamic Time Warping, Neural Networks or the like. In the example implementation of FIG. 2, the speech-to-text converter 228 is shown as part of the controller 220. However, in alternative implementations, the speech-to-text converter 228 may be included in the image capturing device 112.

The input datastore 264 stores a plurality of predetermined inputs that the healthcare provider 120 or an initiator may provide to the electronic input device 116, for example by speaking into the microphone 116A. Example inputs include:

| Input ID | Input |
|---|---|
| 1 | Rehabilitation Center |
| 2 | Physical Therapy |
| 3 | Operation Theater |
| 4 | . . . |

The comparator 232 receives the digital audio 226 from the ADC 224. The comparator 232 compares the digital audio 226 with the inputs stored in the input datastore 264. The comparator 232 determines whether the digital audio 226 matches any one of the inputs stored in the input datastore 264. If the comparator 232 determines that the digital audio 226 matches one of the inputs stored in the input datastore 264, then the comparator 232 sends the input to the message selector 236. The comparator 232 may also send the corresponding ‘Input ID’ to the message selector 236.

If the comparator 232 received the text 226′ from the speech-to-text converter 228, then the comparator 232 may use a SQL (Structured Query Language) query to retrieve inputs that match the text 226′, for example:

SELECT * FROM InputDatastore
WHERE Input=’rehabilitation center’

Alternatively, if the text 226′ is not available then the comparator 226 may compare the digital audio 226 with stored digital audio samples in the input datastore 264. The comparator 232 may compare the amplitudes, the frequencies, the pitch or any other suitable audio characteristic of the digital audio 226 with the digital audio samples stored in the input datastore 264.

In some implementations, the digital audio 226 or the text 226′ may include several phrases. For example, the healthcare provider 120 may say “taking the patient for physical therapy”. In this implementation, the comparator 232 may determine whether the digital audio 226 or the text 226′ contains any one of the stored inputs from the input datastore 264. For example, if the comparator 232 is programmed using Java, then the comparator 232 may determine whether the text 226′ contains one of the stored inputs from the input datastore 264 by using the contains property of a string, for example:

boolean return_value=received_input.contains(stored_input);

The message datastore 268 stores messages corresponding with various inputs. For example, the message datastore 268 stores a message named ‘REHAB’ for inputs that resemble “rehabilitation center”, “rehab center” or “rehab”. As another example the message datastore 268 stores a message called ‘OR’ for inputs that resemble “operational theater”, “operation room”, “surgery”, etc. An example message datastore 268 is shown below:

| Message ID | Message | Inputs | Input ID |
|---|---|---|---|
| 1 | REHAB | “rehabilitation center” | 1 |
| | | “rehab center” | |
| | | “rehab” | |
| 2 | PHYSICAL | “physical therapy” | 2 |
| | THERAPY | “physio” | |
| 3 | OR | “operation” | . . . |
| | | “operation room” | |
| | | “surgery” | |
| 4 | . . . | . . . | . . . |

The message selector 236 selects a message from the message datastore 268 based on the input or input ID received from the comparator 232. The message selector 236 may query the message datastore 268 using SQL for a message that corresponds with the input or input ID received from the comparator 232. In response to the query, the message selector 236 receives a message 242 from the message datastore 268 that corresponds with the digital audio 226 or the text 226′.

The video editor 244 receives a video feed 114 from the image capturing device 112. The video editor 244 also receives the message 242 from the message selector 236. The video editor 244 edits or modifies the video feed 114 to include the message 242. In an example implementation, the video editor 244 superimposes the message 242 on the video feed 114. The video editor sends the modified video feed 114′ to the display device 124. The modified video feed 114′ includes the video feed 114 captured by the image capturing device 112 and the message 242. The display device 124 displays the modified video feed 114′. As shown in FIG. 2, the message 242 is superimposed on the video feed 114. In other implementations, the message 242 may be displayed proximate to the video feed 114 without overlaying the video feed 114. For example, the message 242 may be displayed so that the message 242 abuts the video feed 114 instead of overlapping with the video feed 114.

Although in the example implementation of FIG. 2, the server 200 is shown as being separate from the display device 124, in alternative implementations, the server 200 and the display device 124 may be the same. In the alternative implementations, the image capturing device 112 sends the video feed 114 to the display device 124 and the electronic input device 116 sends the analog audio 118 to the display device 124. The display device 124 may include the controller 220 and the memory 260, so that the display device 124 can perform the features described above in relation to the server 200.

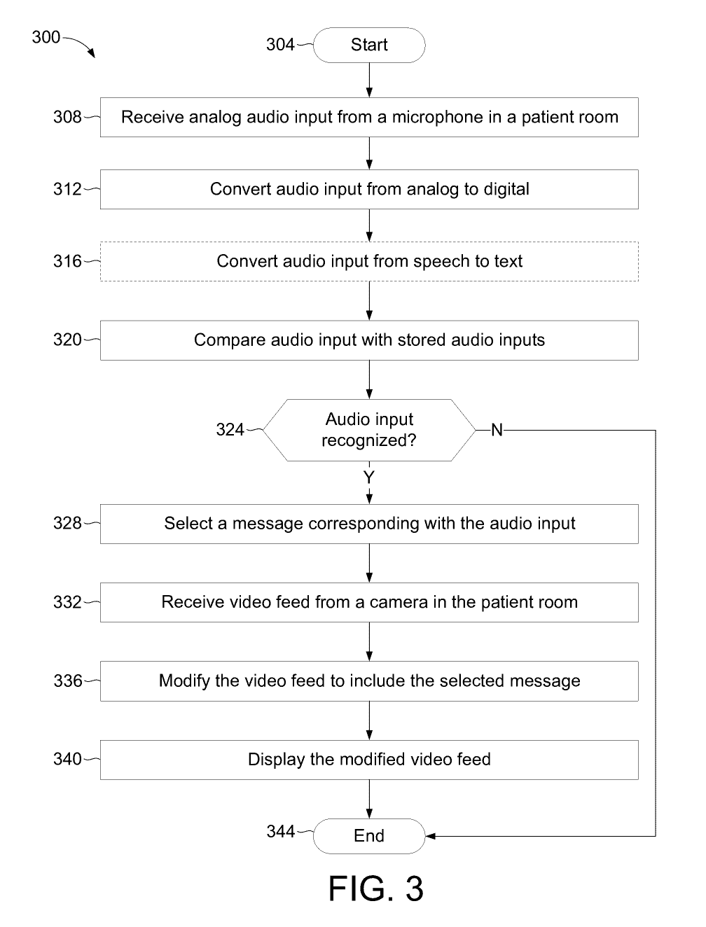

FIG. 3 illustrates a method 300 for monitoring a patient 104. The method 300 starts at 304. At 308, the ADC 224 receives input in the form of analog audio 118 from a microphone 116A in a patient room 108. The ADC 224 converts the analog audio 118 into digital audio 226 (at 312). The speech-to-text converter 228 may convert the digital audio 226 into text 226′ (at 316). The comparator 232 compares the digital audio 226 with stored audio samples in the input datastore 264 (at 320). As explained above, the comparator 232 may compare the digital audio 226 with stored audio samples by comparing various audio characteristics such as amplitude, frequency, pitch or the like. In implementations where the speech-to-text converter 228 has converted the digital audio 226 into text 226′, the comparator 232 may query the input datastore 264 for stored inputs that resemble the text 226′.

The comparator determines whether the digital audio 226 or the text 226′ is a valid input (at 324). In some examples, the healthcare provider 120 may have spoken something into the microphone 116A that is not a valid audio input. For example, the healthcare provider 120 may have activated the microphone 116A inadvertently without the intention of providing any input. If the comparator 232 does not recognize the digital audio 226 or the text 226′ as a valid input, then the method 300 ends at 344, else the method 300 proceeds to 328. At 328, the message selector 326 retrieves a message 242 that corresponds with the audio input from message datastore 268. If the audio input has been converted to text 226′ the message selector 326 queries the message datastore 268 for a message corresponding with the text 226′. As explained above, the message selector 236 may query the message data store 268 using SQL or any other suitable query language.

The video editor 244 receives a video feed 114 from an image capturing device 112 in the patient room 108 (at 332). The video feed 114 may be a continuous video feed or the video feed 114 may be a sequence of images taken periodically. The video editor 244 modifies the video feed 114 to include the selected message 242 (at 336). As explained above, in some implementations, the video editor 244 modifies the video feed 114 by overlaying the message 242 on top of the video feed 114. In other implementations, the video editor 244 appends the message 242 to the video feed 114. The video editor 244 sends a modified video feed 114′ to the display device 124. The display device 124 displays the modified video feed 114′ (at 340). As explained above, the modified video feed 114′ includes the video feed 114 captured by the image capturing device 112 and the message 242 selected by message selector 236. The method 300 ends at 344.

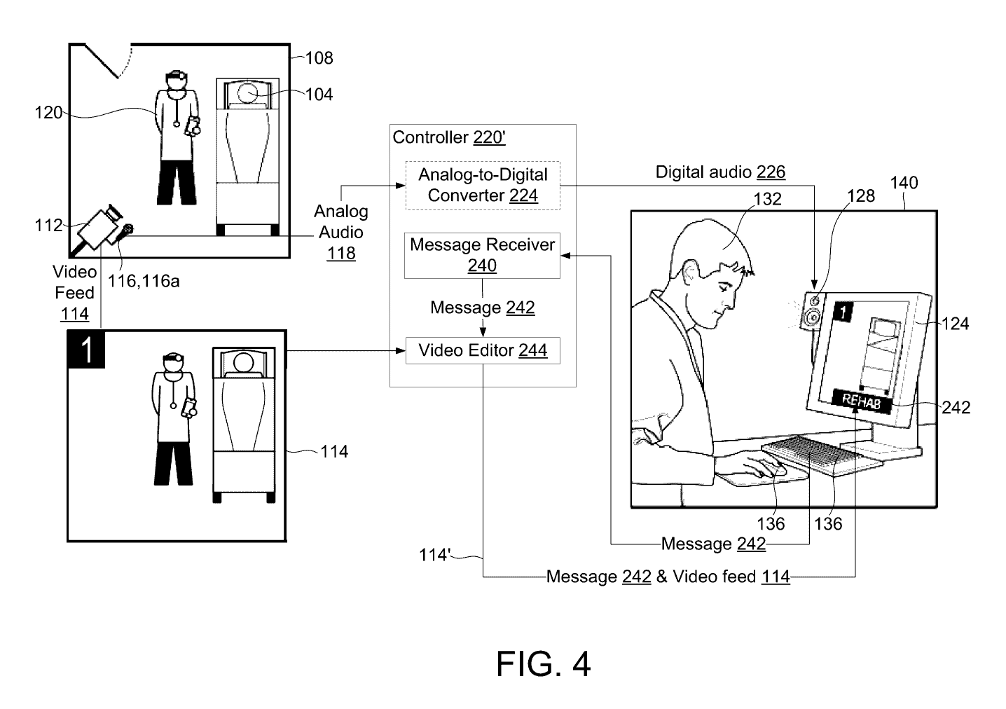

FIG. 4 illustrates another example implementation for monitoring a patient 104 in a patient room 108. The controller 220′ includes the ADC 224, a message receiver 240 and the video editor 244. The controller 220′ receives analog audio 118 from the microphone 116A. The ADC 224 converts the analog audio 118 into digital audio 226. The controller 220′ sends the digital audio 226 to the speaker 128 in the operator room 140. The speaker 128 outputs the digital audio 226 so that the digital audio 226 can be heard by the operator 132 in the operator room 140. Upon hearing the digital audio 226 from the speaker 128, the operator 132 may input a message 242 through the operator device 136. The operator 132 may input the message 242 by typing the message 242. Alternatively, the operator 132 may select the message 242 from a plurality of messages being displayed on the display device 124.

The message receiver 240 receives the message 242 from the operator device 136. The message receiver 240 sends the message 242 to the video editor 244. The video editor 244 edits the video feed 114 to include the message 242. The video editor sends the modified video feed 114′ to the display device 124. The modified video feed 114′ includes the video feed 114 and the message 242. As can be seen in FIG. 4, the display device 124 displays not just the video feed 114 but also the message 242 (‘REHAB’). In this example implementation, the message 242 is superimposed on the video feed 114. However, in other implementations, the message 242 may be displayed adjacent to the video 114 without being superimposed thereon. By displaying the message 242 on the display device 124 in association with the video feed 114, the operator 132 will know why the patient 104 is not in the patient room 108 thereby reducing the likelihood of false missing patient alerts generated by the operator 132. As shown, the display device 124 displays the message ‘REHAB’, so that the operator 132 knows that the patient is in rehab. Therefore, the operator 132 will not likely raise a missing patient alert upon seeing an empty bed in the patient room 108.

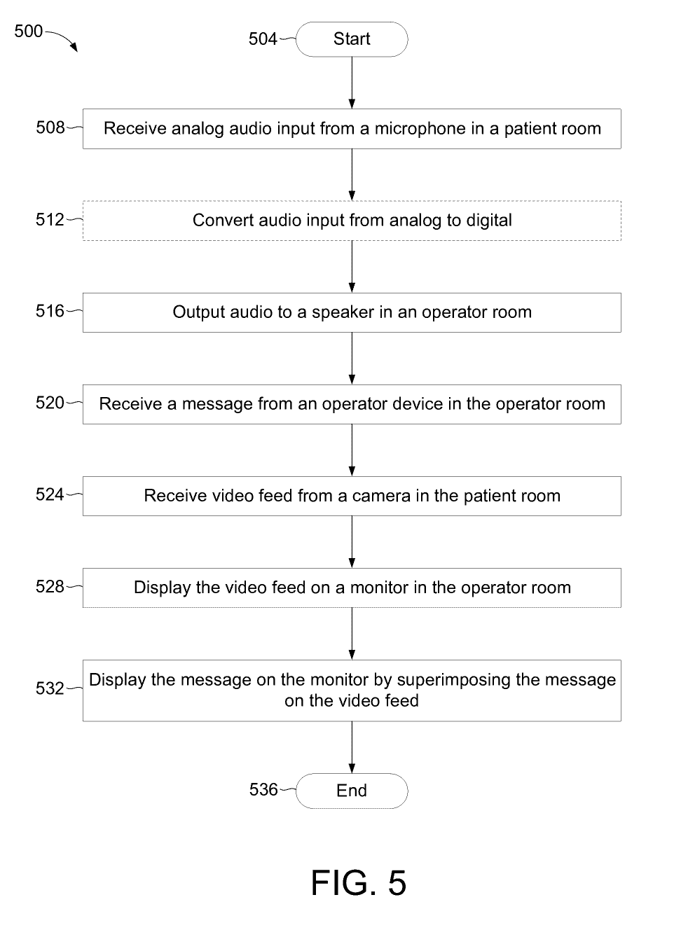

FIG. 5 illustrates an example method 500 for monitoring a patient 104. The method 500 starts at 504. At 508, the controller 220′ receives input in the form of analog audio 118 from a microphone 116A in a patient room 108. The ADC 224 may convert the analog audio 118 into digital audio 226 (at 512). The speaker 128 outputs the analog audio 118 or the digital audio 226 (at 516). At 520, the message receiver 240 receives a message 242 from the operator device 136 in the operator room 140. At 524, the video editor 244 receives a video feed 114 from an image capturing device 112 in a patient room 108. The display device 124 displays the video feed 114 in the operator room 140 (at 528). The display device 124 additionally displays the message 242 on the monitor 124 by superimposing the message 242 on the video feed 114. As explained above, the video editor 244 may modify the video feed 114 to include the message 242. The method 500 ends at 536.

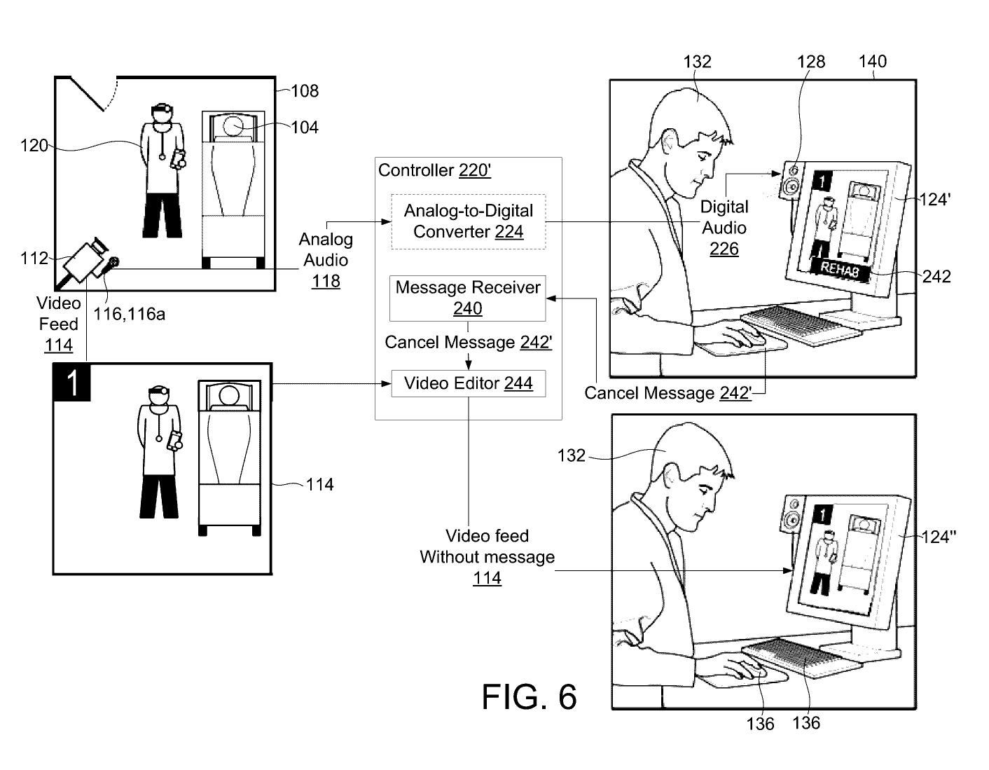

FIG. 6 illustrates another example implementation for monitoring a patient 104 in a patient room 108. In this example the display device 124′ is showing that the patient is in ‘REHAB’ by displaying the message 242. The healthcare provider 120 provides audio input to the microphone 116A. The speaker 128 outputs the audio input so that the operator 132 can hear the healthcare provider 120. In this example the healthcare provider 120 can indicate that the patient has been brought back from rehab. Upon hearing the audio the operator 132 may use the operator device 136 to cancel the message 242. The operator 132 may send a cancellation message 242′ to a message receiver 240 of the controller 220′. The message receiver 240 receives the cancellation message 242′ from the operator device 136. The message receiver 240 forwards the cancellation message 242′ or instructs the video editor 244 to remove the message 242 from the display device 124. The video editor 244 modifies the video feed 114 so that the message 242 is not being displayed on the display device 124. As shown, the display device 124″ displays the video feed 114 but not the message 242. In this example implementation, a message that was previously sent when a patient was being taken out of his room for ‘REHAB’ can be canceled when the patient is returned to the room.

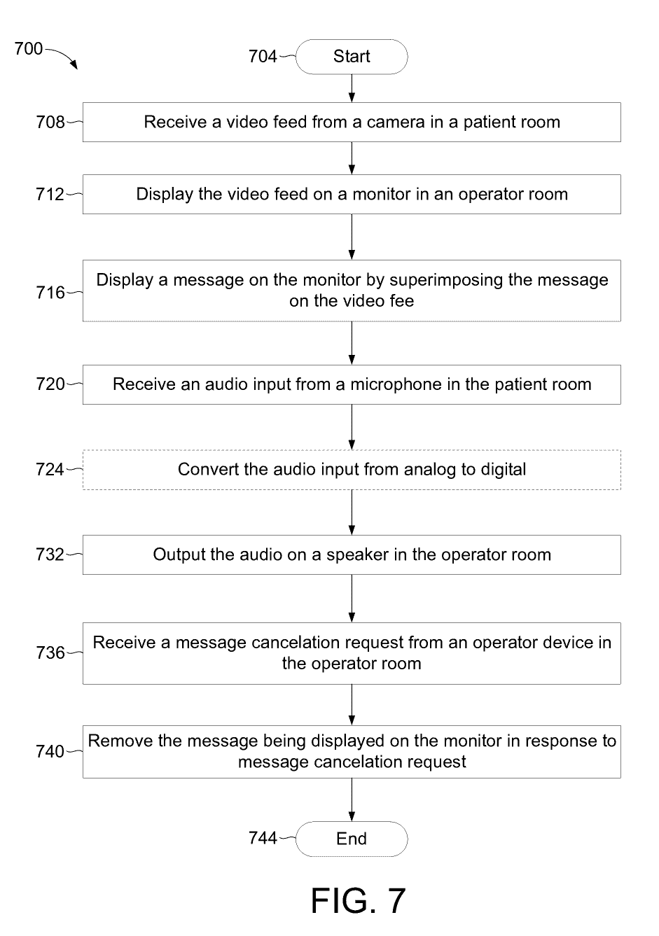

FIG. 7 illustrates an example method 700 for canceling a message 242. The method 700 starts at 704. At 708, the video editor 244 receives the video feed from an image capturing device 112 in a patient room 108. The display device 124 displays the video feed 114 in the operator room 140 (at 712). The monitor 124 additionally displays the message 242 by superimposing the message 242 on the video feed 114 (at 716). At 720, the controller 220′ receives an input in the form of analog audio 118 from the microphone 116A in the patient room 108. The ADC 224 may convert the analog audio 118 into the digital audio 226 (at 724). The speaker 128 outputs the analog audio 118 or the digital audio 226 (at 732) so that the analog audio 118 or the digital audio 226 can be heard by the operator 132 presiding in the operator room 140.

At 736, the message receiver 240 receives a cancellation message 242′ from an operator device 136 in the operator room 140. The message receiver 240 instructs the video editor 244 to remove the message 242 that is being displayed on display device 124 (at 740). The video editor 244 edits the video feed 114′ so that the message 242 is not being displayed on the display device 124″. The method 700 ends at 744.

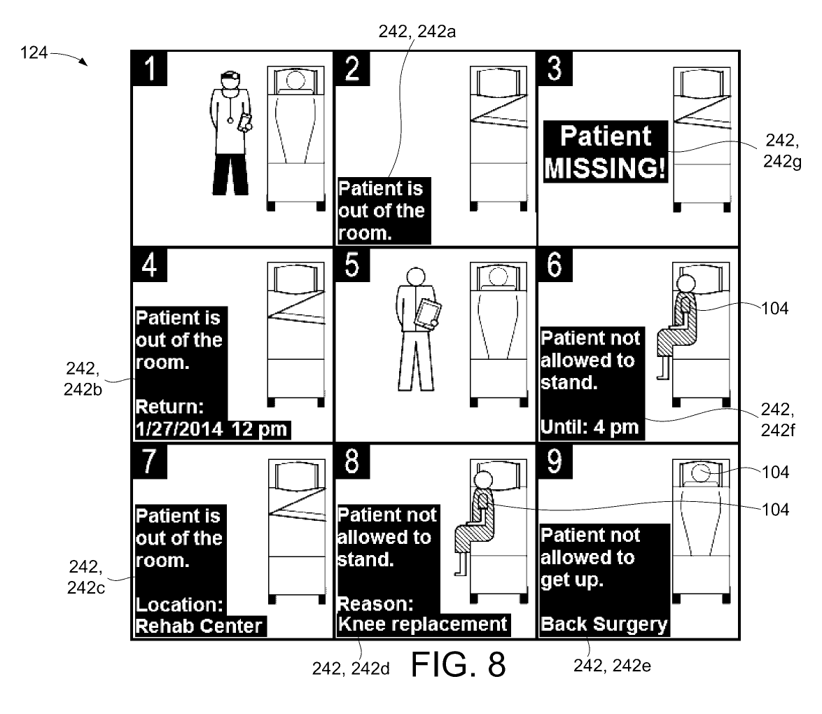

FIG. 8 depicts a display device 124 with messages 242. Message 242A states that the patient is out of the room. As explained above, the message 242A could have been selected by the operator 132 via the operator device 136 or the message 242A could have been automatically selected when the healthcare provider 120 scanned his RFID tag against the RFID scanner 116B. Message 242B not only shows that the patient is out of the room but also shows when the patient is expected to return. The return time can be provided by the healthcare provider 120 through the electronic input device 116. For example, the healthcare provider 120 can say the return time into the microphone 116A. Alternatively, the healthcare provider 120 can enter the expected return time into the computer terminal 116C, the mobile phone 116D or the tablet computer 116E. Advantageously, the message 242B not only tells the operator 132 that the patient is out of the room but also when the patient is expected to return.

Message 242C displays that the patient is out of the room. Additionally, the message 242C displays a location of where the patient is currently located. In this example, the location is ‘Rehab Center’. The location can be provided by the healthcare provider 120 through the electronic input device 116. For example the healthcare provider 120 could have stated the location verbally through the microphone 116A. Alternatively, the healthcare provider 120 could have provided a location through the computer terminal 116C, the mobile phone 116D or the tablet computer 116E. Advantageously, the message 242C not only tells the operator 132 that the patient is out of the room but also where the patient is currently located. So, if a healthcare provider or the patient's family member needs to locate the patient, the healthcare provider or the family member may contact the operator 132 in the operating room 140 and the operator 132 may communicate the location of the patient with help from the message 242C.

A message 242D displays a medical restriction. In this example, the patient 104 is “not allowed to stand”. The patient 104 is in the patient room 108 but the patient has a medical restriction (cannot stand) due to a condition (knee replacement surgery). The healthcare provider 120 can provide a patient condition thorough the electronic input device 116. For example, the healthcare provider 120 can verbally state through the microphone 116A that the patient 104 has had knee surgery and that the patient 104 is “not allowed to stand”. Advantageously, the message 242D not only allows the operator 132 to make sure that the patient stays within the room but also helps in ensuring that the patient 104 does not do anything that is inconsistent with a medical condition of the patient. In this example, the patient has had a knee replacement surgery and the patient is not allowed to stand. The operator 132 can alert a healthcare provider 120 if the patient 104 stands. Similarly, a message 242E can state a medical condition that the patient has had back surgery and that the patient is not allowed to get up. The operator 132 can alert a healthcare provider 120 if the patient 104 attempts to get up thereby preventing further injury to the patient 104.

A message 242F states a medical restriction that the patient is not allowed to stand and also states a time until when that medical restriction persists. In this example, the patient is not allowed to stand until 4:00 p.m. A message 242G states that the patient is missing from the room. The message 242G can be generated automatically, for example, if the controller 220 can sense that the patient 104 in not in the room through image recognition and a message 242 has not been selected by the message selector 236 or received by the message receiver 240. The controller 220 can therefore infer that the patient is not accounted for and the controller 220 can generate the message 242G which tells the operator 132 that the patient is missing. Advantageously, the operator 132 can raise an alert and attempt to find the missing patient.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Implementations of the subject matter and the functional operations described in this specification can be implemented in digital electronic circuitry, or in computer software, firmware, or hardware, including the structures disclosed in this specification and their structural equivalents, or in combinations of one or more of them. Moreover, subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The terms “data processing apparatus”, “computing device” and “computing processor” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as an application, program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

One or more aspects of the disclosure can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

A number of implementations have been described. Nevertheless, it will be understood that various modifications may be made without departing from the spirit and scope of the disclosure. Accordingly, other implementations are within the scope of the following claims.

1-23. (canceled)

24. A method for monitoring a patient, the method comprising:
receiving, at a controller, an electronic signal encoded with an image, of a patient room, from an image capturing device;
displaying the image of the patient room on a display device;
receiving, via a microphone, an electronic analog signal which carries speech information regarding a status associated with a patient in the patient room;
utilizing an analog-to-digital converter for converting the electronic analog signal to an electronic digital signal;
utilizing a speech-to-text converter for converting the speech information into text;
utilizing a comparator for
comparing the digital signal with stored samples in an input datastore, and
determining whether the digital signal or the converted text is a valid input, and,
if the comparator determines that the digital signal or the text is a valid input,
utilizing an alphanumeric message selector for selecting an alphanumeric message based on the status associated with the patient from a plurality of alphanumeric messages; and
utilizing a video editor for modifying the image by displaying the selected alphanumeric message.

25. The method of claim 24, wherein the status associated with the patient in the patient room includes:
a diagnosis of the patient,
protected health information of the patient,
a description of health of the patient, or
restrictions imposed on the patient.

26. The method of claim 24, wherein the status associated with the patient in the patient room includes:
an out-of-room location of the patient.

27. The method of claim 26, wherein the out-of-room location of the patient includes:
a rehabilitation center room,
a physical therapy room, or
a surgery room.

28. The method of claim 27, wherein the out-of-room location of the patient includes:
an indicator indicating that removal of the patent from the patient room was authorized by a healthcare provider.

29. The method of claim 27, wherein the out-of-room location of the patient includes:
an indicator indicating that removal of the patent from the patient room was not authorized by a healthcare provider.

30. An apparatus for monitoring a patient, the apparatus comprising:
an image capturing device;
an electronic input device;
a display device; and
means for:
receiving an electronic signal encoded with an image of a patient room captured by the image capturing device,
displaying the image of the patient room on the display device,
receiving, by way of the electronic input device, an electronic input signal regarding a status associated with the patient in the patient room, and
displaying an alphanumeric message on the display device based on the status of the patient.

31. The apparatus of claim 30, wherein the electronic input signal regarding the status associated with the patient comprises a time identifier; and wherein the alphanumeric message displayed on the display device comprises alphanumeric characters associated with the time identifier.

32. The apparatus of claim 30, wherein the electronic input signal regarding the status associated with the patient comprises a location; and wherein the alphanumeric message displayed on the display device comprises alphanumeric characters indicating the location.

33. The apparatus of claim 30, wherein the alphanumeric message displayed on the display device comprises alphanumeric characters indicating the status of the patient.

34. The apparatus of claim 30, wherein the electronic input device receives an authentication signal.

35. The apparatus of claim 30, wherein the electronic input device comprises a microphone.

36. The apparatus of claim 30, wherein the electronic input device comprises a mobile electronic communication device.

37. The apparatus of claim 30, wherein the electronic input device comprises a computer terminal.

38. The apparatus of claim 30, further comprising an input datastore storing a plurality of electronic input signals; and wherein the electronic input device receives a selection of one of the plurality of electronic input signals as the electronic input signal regarding the status associated with the patient.

39. The apparatus of claim 30, further comprising an alphanumeric message datastore storing a plurality of alphanumeric messages, each alphanumeric message corresponding with one or more electronic input signals; and wherein the display device displays the alphanumeric message that corresponds with the electronic input signal received by the electronic input device.

40. A method for monitoring a patient, the method comprising:
receiving, at a controller, an electronic signal encoded with an image, of a patient room, from an image capturing device;
displaying the image of the patient room on a display device;
receiving, via an electronic input device, an electronic input signal regarding a status associated with a patient in the patient room; and
displaying an alphanumeric message on the display device based on the status associated with the patient.

41. The method of claim 40, wherein receiving the electronic input signal regarding the status associated with the patient comprises receiving a time identifier; and wherein displaying the alphanumeric message comprises displaying alphanumeric characters associated with the time identifier.

42. The method of claim 40, wherein receiving the electronic input signal regarding the status associated with the patient comprises receiving a location; and wherein displaying the alphanumeric message comprises displaying alphanumeric characters indicating the location.

43. The method of claim 40, wherein displaying the alphanumeric message comprises displaying an indicia of the status of the patient.

44. The method of claim 40, further comprising authenticating the electronic input signal.

45. The method of claim 40, wherein the electronic input device comprises a microphone; and wherein receiving the electronic input signal regarding the status associated with the patient comprises receiving audio input via the microphone.

46. The method of claim 40, wherein the electronic input device comprises a mobile electronic communication device; and wherein receiving the electronic input signal regarding the status associated with the patient comprises receiving the electronic input signal via the mobile electronic communication device.

47. The method of claim 40, wherein the electronic input device comprises a computer terminal; and wherein receiving the electronic input signal regarding the status associated with the patient comprises receiving the electronic input signal via the computer terminal.

48. The method of claim 40, further comprising storing a plurality of electronic input signals in an input datastore; and wherein receiving the electronic input signal regarding the patient comprises receiving a selection of one of the plurality of electronic input signals as the electronic input signal regarding the patient.

49. The method of claim 40, further comprising storing a plurality of alphanumeric messages in an alphanumeric message datastore, each alphanumeric message corresponding with one or more electronic input signals; and wherein displaying the alphanumeric message comprises displaying one of the plurality of alphanumeric messages that corresponds with the electronic input signal received by the electronic input device.

50. An apparatus for monitoring a patient, the apparatus comprising:
a video camera;
a microphone;
a monitor;
an alphanumeric message datastore storing a plurality of alphanumeric messages; and
means for:
receiving a video feed of a patient room from the video camera,
displaying the video feed of the patient room on the monitor,
receiving, via the microphone, an electronic input which carries audio information regarding a patient in the patient room from a healthcare provider,
selecting, from the alphanumeric message datastore, one of the plurality of alphanumeric messages corresponding with the audio information, and
displaying the selected alphanumeric message on the monitor in association with the video feed of the patient room being displayed on the monitor.

51. The apparatus of claim 50, wherein the selected alphanumeric message is overlaid on top of the video feed of the patient room being displayed on the monitor.

52. The apparatus of claim 50, wherein the monitor displays a plurality of video feeds, each video feed being from a different patient room; and the monitor further displays a plurality of alphanumeric messages, each alphanumeric message being displayed in associated with a corresponding video feed.

53. The method of claim 50, wherein the means for receiving, via the microphone, includes means for receiving an analog electronic signal from the microphone, further including utilizing an analog-to-digital converter for converting the analog electronic signal to a digital electronic signal.

54. The method of claim 53, wherein the analog signal includes speech information carried thereon, wherein the method further comprises utilizing a speech-to-text converter for converting the speech into text.

55. The method of claim 54, further comprising utilizing a comparator for comparing the digital signal information with stored information samples in an input datastore.

56. The method of claim 54, further comprising utilizing the comparator for determining whether the digital signal or the text is a valid input, if the comparator recognizes the digital signal or the text, an alphanumeric message selector selects an alphanumeric message from a plurality of alphanumeric messages that corresponds with the digital signal from an alphanumeric message datastore.

57. The method of claim 56, wherein the displaying the alphanumeric message on the display device includes utilizing a video editor for modifying the image to include the selected alphanumeric message.

58. The method of claim 57, further comprising utilizing the display device for displaying a modified image that includes the image captured by the image capturing device and the selected alphanumeric message.

59. The method of claim 53, further comprising utilizing a speaker located at a remote operator room for outputting the analog signal or the digital signal audibly.

60. The method of claim 59, further comprising sending from the electronic input device located at the remote operator room the alphanumeric message to an alphanumeric message receiver and then receiving at a video editor the image from the image capturing device that was captured in a patient room and then further utilizing the display device for displaying the image in the remote operator room while also superimposing the alphanumeric message on the image.

61. The method of claim 59, further comprising sending from the electronic input device located at the remote operator room a cancellation message to an alphanumeric message receiver and instructing a video editor to remove the alphanumeric message that is displayed on the display device.
